(19) United States
(12) Patent Application Publication
(10) Pub. No.: US 2003/0125314 A1
(43) Pub. Date: Jul. 3, 2003

(54) Photodynamic therapy for the treatment of epilepsy

(76) Inventor: Edie Zusman, Piedmont, CA (US)

Correspondence Address: GRAY CARY WARE & FREIDENRICH LLP, 153 TOWNSEND, SUITE 800, SAN FRANCISCO, CA, 94107, US

(21) Appl. No.: 10/309,946

(22) Filed: Dec. 3, 2002

Related U.S. Application Data
(60) Provisional application No. 60/336,955, filed on Dec. 3, 2001.

(51) Int. Cl. A61K 31/555; A61N 1/30; A61K 31/409; A61K 31/198

(52) U.S. Cl. 514/185; 514/410; 604/20; 514/561

(57) ABSTRACT

The present invention is based on the discovery that cells associated with seizure have selective intake of photoactive compounds. The present invention provides methods of triggering cell death in cells associated with seizure conditions by exposing such cells to photoactive compounds and irradiating the photoactive compounds contained within the cells. The present invention also provides methods of labeling cells associated with seizure conditions by exposing such cells with photoactive compounds. In addition, the present invention provides model systems useful for studying seizure conditions.

### RELATED APPLICATIONS

The present application claims priority under 35 U.S.C. §119(e) from provisional application No. 60/336,955, filed Dec. 3, 2001.

### FIELD OF THE INVENTION

This invention relates generally to the field of photodynamic therapy, and more specifically to the use of photodynamic therapy to treat a seizure condition. In addition, the present invention relates to the use of photoactive compounds to label cells associated with a seizure condition.

### BACKGROUND OF THE INVENTION

For more than 2,000,000 people with epilepsy the daily life challenges are well known—decreased school and work performance, medication side effects, difficulty or inability to obtain a driver's license, psychosocial problems and fear of injury or sudden death. Today surgery is usually the only known “cure” for epilepsy. Some patients are candidates for restrictive surgery, however in most cases, the epileptic focus cannot be clearly localized or visualized. Although the use of subdural electrodes and intraoperative EEG can approximate the location of the epileptic focus, the surgery still has many side effects, e.g., the operation can removes tissues that are not specific to the epileptic process while significant for maintaining normal human functions.

There is a need in the art to provide methods useful for treating seizure, especially epileptic seizure conditions. There is also a need in the art to provide methods for identifying or localizing cells associated with seizure conditions.

### SUMMARY OF THE INVENTION

The present invention is based on the discovery that cells associated with a seizure condition uptake photoactive compounds, e.g., more actively than normal cells. Accordingly the present invention provides methods of reducing cells associated with seizure conditions by exposing the cells to photoactive compounds and triggering cell death in cells that up-take photoactive compounds. In addition, the present invention provides methods of labeling cells associated with seizure conditions by using photoactive compounds.

In one embodiment, the present invention provides a method of triggering a cell death process in a cell associated with a seizure condition. The method includes exposing the cell to a photoactive compound and irradiating the cell with an energy source comprising at least one wavelength capable of being absorbed by the photoactive compound, whereby triggering a cell death process in the cell.

In another embodiment, the present invention provides a method of treating a seizure condition. The method includes administering to a subject in need of such treatment an effective amount of a photoactive compound and irradiating cells associated with the seizure condition with an energy source comprising at least one wavelength capable of being absorbed by the photoactive compound.

In yet another embodiment, the present invention provides a method of labeling a cell associated with a seizure condition. The method includes exposing a population of cells to a photoactive compound, whereby a cell associated with a seizure condition is labeled by up-taking the photoactive compound.

In still another embodiment, the present invention provides a kit which includes a photoactive compound and an instruction for using the photoactive compound to trigger a cell death process in cells associated with a seizure condition.

In another embodiment, the present invention provides a kit which includes a photoactive compound and an instruction for labeling cells associated with a seizure condition.

In yet another embodiment, the present invention provides a neural model system useful for studying a seizure condition wherein cells associated with the seizure condition are selectively reduced by being exposed to a photoactive compound and irradiated by an energy source comprising at least one wavelength capable of being absorbed by the photoactive compound.

### SUMMARY OF THE FIGURES

FIG. 1 shows the results obtained from the Morris Water Maze study.

FIG. 2 shows the results obtained from Incline Plane study.

FIG. 3 shows the intracellular PpIX content across experimental groups. In each group, coronal sections through hippocampus contain more PpIX than sections through the frontal cortex. The animals that were kindled have a higher average PpIX content than the control. Animals who were kindled and seizure-induced have the highest average PpIX as measured by spectrofluorometry.

### DETAILED DESCRIPTION

The present invention relates in general to using photoactive compounds in labeling or inactivating cells associated with seizure conditions. The present inventions provides methods and kits useful for reducing, more specifically selectively triggering cell death in cells associated with seizure conditions by irradiating photoactive compounds up-taken by these cells. In addition, the present invention provides methods and kits for labeling cells associated with seizure conditions by using photoactive compounds. The present invention also provides cellular, tissue, or animal model systems useful for studying seizure conditions.

According to the present invention, cells associated with seizure conditions, e.g., human neural cells can be inactivated or eliminated by exposing the cells to photoactive compounds and irradiating the cells with an energy source with at least one wavelength capable of being absorbed by the photoactive compound. Usually cells associated with seizure conditions have a more active or preferential uptake of photoactive compounds than normal cells. Irradiation of cells containing photoactive compounds generally causes production of cytotoxic photoproducts in the cells, e.g., singlet oxygen species which can trigger a cell death process including apoptosis of cells and tissue necrosis.

In one embodiment, a locus of cells or a tissue region suspecting of containing cells associated with seizure conditions in a subject is exposed to photoactive compounds and then be irradiated with an energy source including at least one wavelength absorbed by the photoactive compounds. Usually each episode of a seizure condition enhances the uptake of photoactive compounds of the cells associated with the seizure condition, therefore in another embodiment, a locus of cells or a tissue region suspecting of containing cells associated with seizure conditions in a subject is exposed to photoactive compounds and the cells are irradiated after undergoing at least one episode of seizure conditions.

In general, exposing cells associated with seizure conditions to photoactive compounds can be carried out by any suitable means available to one skilled in the art. For example, cells or tissue samples containing such cells can be incubated in vitro with photoactive compounds or a medium containing photoactive compounds. Alternatively a subject, e.g., human can be administered with photoactive compounds or compositions thereof The photoactive compounds can be administered through various suitable route, e.g., oral, intravenous, or topical administration.

In addition, one or more loci of cells or tissue regions suspecting of containing cells associated with seizure conditions can be exposed to photoactive compounds in situ, using in vivo drug delivery devices capable of slow localized releasing of photoactive compounds.

The photoactive compound used in the present invention can be any known or later discovered compounds suitable for photodynamic therapy, e.g., capable of absorbing photons of light and transfer that energy to oxygen which then converts to a cytotoxic or cytostatic species. A list of representative classes of photoactive compounds, e.g., photosensitizers commonly used for photodynamic therapy are provided in Table 1.

TABLE 1
Reactive oxygen producing photosensitizer molecules and
light emitting photosensitive molecules.

Pyrrole-derived macrocyclic compounds
Naturally occurring or synthetic porphyrins and derivatives thereof
Naturally occurring or synthetic chlorines and derivatives thereof
Naturally occurring or synthetic bacterio-chlorins and derivatives thereof
Synthetic isobacteriochlorins and derivatives thereof
Phthalocyanines and derivatives thereof
Naphthalocyanines and derivatives thereof
Porphycenes and derivatives thereof
Porphycyanines and derivatives thereof
Pentaphyrin and derivatives thereof
Sapphyrins and derivatives thereof
Texaphyrins and derivatives thereof
Phenoxazine dyes and derivatives thereof
Phenothiazines and derivatives thereof
Chalcoorganapyrylium dyes and derivatives thereof
Triarylmethanes and derivatives thereof
Rhodamines and derivatives thereof
Fluorescenes and derivatives thereof
Azaporphyrins and derivatives thereof
Benzochlorins and derivatives thereof
Purpurins and derivatives thereof
Chlorophylls and derivatives thereof
Verdins and derivatives thereof

In one embodiment, the photoactive compound of the present invention is any photosensitizer preferentially up-taken by neoplastic cells or tissues, e.g., photosensitizers suitable for treating neoplasia, especially brain tumors. In another embodiment, the photoactive compound of the present invention is a photosensitizer capable of passing through blood brain barrier.

In yet another embodiment, the photoactive compound of the present invention is a member of 5-aminolevulinic acid (ALA), 5-aminolevulinic acid-induced compounds, e.g., protoporphyrin IX (PpIX), Photofrin, chloroaluminium phthalocyanine, Tin Ethyl Etiopurpurin, and meta-tetra (hydroxyphenyl)chlorin.

In still another embodiment, the photoactive compound of the present invention is a photosensitizer absorbing energy at a wavelength that is capable of being transmitted through tissues or bone structures, e.g., human skull.

The photoactive compound used in the present invention can also be derivatives of any known or later discovered photoactive compounds. In one embodiment, the derivatives of photoactive compounds include, without limitation, precursors and metabolites of any photoactive compounds, and any other modifications that facilitate the preferential uptake of these compounds by cells associated with seizure conditions. For example, as part of the heme biosynthetic pathway the ubiquitous intracellular compound, ALA is converted to the photoactive molecule, PpIX; and excess exogenous ALA bypasses the rate-limiting step of the heme pathway and leads to an excess intracellular accumulation of PpIX, which can be photoactivated by an energy source.

The energy source used in the present invention to irradiate the photoactive compounds contained within cells can be any suitable energy sources that contain at least one wavelength that can be absorbed by the photoactive compound to be irradiated. For example, the energy source can be a laser that emitting energy at a wavelength that is absorbed by the photoactive compound to be irradiated.

The irradiation of the photoactive compounds contained within cells associated with seizure conditions can be carried out through various means known to one skilled in the art. For example, the irradiation can be performed by sending an energy of the appropriate wavelength to a locus of cells or tissue region suspecting of or being identified as a locus for cells associated with seizure conditions, e.g., coronal sections through hippocampus in a human brain.

In one embodiment, the irradiation is carried out by less invasive procedures, e.g., using a device including a monochromatic light source such as laser, the light output of which may be coupled to a light delivery catheter for conduction and delivery to a remote target tissue. Such interventional light delivery catheters are well known in the art and are described, for example, in U.S. Pat. Nos. 5,169,395, 5,196,005, and 5231684.

In another embodiment, the irradiation can be carried out in conjunction with other devices, especially devices useful for exposing the cells to photoactive compounds of the present invention. For example, drug delivery devices and/or a balloon perfusion catheter and/or various medicament-dispensing stents for the slow localized release of the photoactive compounds can be used in connection with a light source and light delivery catheter.

In yet another embodiment, the irradiation is carried out through a surgical procedure, e.g., trephination, craniotomy, or endoscopy by surgically exposing the area to be irradiated directly to an energy source with minimum obstruction of other tissues or structures. In still another embodiment, the irradiation is carried out through a non-surgical procedure, e.g., by exposing a region suspecting of or being identified as a locus for cells associated with epilepsy to an energy source without surgically removing tissues or structures between the cells to be irradiated and the energy source.

The methods provided by the present invention to trigger cell death in cells associated with seizure conditions can be used in various situations where there is a need to inactivate or eliminate cells associated with seizure conditions, e.g., treating or preventing a seizure condition.

Seizure conditions usually include a brain seizure or convulsion, e.g. abrupt alteration in cortical electrical activity manifested clinically by a change in consciousness or by a motor, sensory, or behavioral symptom. Seizure conditions usually include epileptic seizure, e.g., recurrent seizures present over months or years, often with a stereotyped clinical pattern. In one embodiment, seizure conditions include partial or focal seizures, e.g., simple or complex partial seizures or secondary generalized partial seizures. In another embodiment, seizure conditions include primary generalized seizures, e.g., tonic clonic, absence, myoclonic, and atonic or akinetic.

Cells associated with seizure conditions are usually a collection of neurons in the brain that involved in alteration in cortical electrical activity, e.g., sudden electrical discharge. According to the present invention, cells associated with seizure conditions can be any cells involved in a seizure condition or any neurological conditions associated with sudden electrical discharge of neurons. Cells associated with seizure conditions can be in a human subject, a tissue sample, a tissue culture, or an animal model.

According to one aspect of the present invention, the methods provided by the present invention can be used to treat a subject, e.g., a human with a seizure condition. More specifically, an effective amount of the photoactive compounds of the present invention can be administered to a subject, e.g., human with a seizure condition and cells suspected of or identified as locus of cells associated with seizure conditions, e.g., coronal sections through hippocampus in brain can be irradiated by an energy source containing at least one wavelength absorbed by the photoactive compounds.

Photoactive compounds of the present invention can be provided as a composition including one or more other non-active ingredients, e.g., ingredients that do not interfere with the function of the active ingredients. For example, the composition containing one or more photoactive compounds of the present invention can include a suitable carrier or be combined with other therapeutic agents.

A suitable carrier can be an aqueous carrier including any safe and effective materials for use in the compositions of the present invention. A suitable carrier can also be a pharmaceutically acceptable carrier that is well known to those in the art.

Pharmaceutically acceptable salts can also be used in the composition, for example, mineral salts such as sodium or stannous fluorides, or sulfates, as well as the salts of organic acids such as acetates, proprionates, carbonates, malonates, or benzoates. The composition can also contain liquids, e.g., water, saline, glycerol, and ethanol, as well as substances, e.g., wetting agents, emulsifying agents, or pH buffering agents.

In generally, an effective amount of the photoactive compounds of the present invention to be administered can be determined on a case-by-case basis. Factors to be considered usually include age, body weight, stage of the condition, other disease conditions, duration of the treatment, and the response to the initial treatment.

Typically, the photoactive compounds of the present invention are prepared as a topical or an injectable, either as a liquid solution or suspension. However, solid forms suitable for solution in, or suspension in, liquid vehicles prior to injection can also be prepared. The composition can also be formulated into an enteric-coated tablet or gel capsule according to known methods in the art.

The photoactive compounds of the present invention may be administered in any way which is medically acceptable which may depend on the condition being treated. Possible administration routes include injections, by parenteral routes such as intravascular, intravenous, or others, as well as oral, nasal, ophthalmic, topical, or pulmonary, e.g., by inhalation. The compositions may also be directly applied to tissue surfaces. Sustained release, pH dependent release, or other specific chemical or environmental condition mediated release administration is also specifically included in the invention, by such means as depot injections or implants.

Usually irradiation of the cells in a subject taken photoactive compounds can be carried out as soon as the cells to be irradiated have up taken the photoactive compounds, e.g., either concurrently or subsequently to the administration of the photoactive compounds to the subject. Several suitable means can be used to monitor the uptake of photoactive compounds by the cells of interest in a subject. For example, one can administer a photoactive compound in combination with a minor amount of a tracer, e.g., the photoactive compound labeled with a radioactive isotope to monitor the concentration of the radioactive tracer in cells associated with seizure conditions and determine the optimal time for beginning irradiation of the target cells or regions of brain tissues.

In one embodiment, an effective amount of the photoactive compounds of the present invention is administered to a human subject and irradiation is not carried out until such human subject has experienced at least one episode of a seizure condition, e.g., seizure.

According to yet another aspect of the present invention, photoactive compounds can be used to label or signify, specifically, cells associated with seizure conditions. For example, photoactive compounds can be administered to a subject in need of such procedure and be preferentially up-taken by cells associated with seizure conditions, e.g., the cells associated with seizure conditions are labeled or signified by photoactive compounds contained therein.

Cells labeled with photoactive compounds can be useful for various purposes, e.g., to selectively inactivate or eliminate the cells by irradiating the photoactive compounds contained therein or to detect the cells associated with seizure conditions by detecting the photoactive compound contained therein. In general, photoactive compounds can be detected either directly or through products induced by the photoactive compounds or entities conjugated with the photoactive compounds.

In one embodiment, cells are exposed to photoactive compounds conjugated with a detectable entity, e.g., a fluorescent label and are visualized through a fluorescent filter. In another embodiment, cells are exposed to photoactive compounds conjugated with an imaging entity, e.g., a radioactive isotope and are visualized through readily available imaging devices.

Detecting cells associated with seizure conditions using the methods provided by the present invention can be useful for various applications. For example, such methods can be used to label cells associated with seizure conditions in a human subject right before or during surgical operations, e.g., to facilitate surgical removal of cells associated with seizure conditions. Such methods can also be used to label cells associated with seizure conditions in a neural cellular tissue culture, a brain slice model, or an animal model for studying seizure conditions.

Another feature of the present invention provides a kit containing one or more photoactive compounds and an instruction for using the photoactive compounds to label or triggering cell death in cells associated with seizure conditions, e.g., in a tissue culture, a tissue sample, an animal model, or a human subject, according to the methods provided by the present invention.

The present invention also provides a neural model system useful for studying seizure conditions. Such model system can be a cellular tissue culture, brain slice model, or an animal model where cells associated with seizure conditions have been labeled or reduced by the methods provided by the present invention.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

In the following experiments, we present a novel application of a technique. It is called photodynamic therapy (PDT) and is a two part process which insures specificity and selectivity of treatment. The first part of the process involves selective uptake of a photo-active compound into “epileptic” neurons (i.e., cells that contribute to seizure generation) within the brain region of interest. The second step targets laser light to that specific brain (in our studies, the hippocampus) activating the photosensitizing agent and initiating a cell death process.

We have employed the drug, 5-aminolevulinic acid (ALA) which already has FDA approval so that if this approach shows safety and efficacy in animal models, it can easily be transferred to patient use.

We have assessed this therapy in kindled rats. Kindling is a well characterized and reproducible model for reflecting epileptogensis, the process by which a normal brain region becomes an epileptic focus. Animals are kindled with an electrode implanted into the perforant path region of the brain. Each day the animals receive a small stimulus. Initially no seizure activity is induced, but over two or three weeks, this same low level of stimulation generates increasing seizure activity until an animal has 3 consecutive tonic clonic (Stage 5) seizures. Once fully kindled, animals retain this seizure sensitivity even if not stimulated for days or months.

Using this model of epilepsy, we have tested the efficacy of photodynamic therapy. We have conducted several studies to assess the multiple variables of this new therapy. We have evaluated safety using behavioral paradigms and other activity measures. We have seen evidence of apoptotic cell death histologically following this new treatment. The animals tested for kindling state following PDT, have thus far demonstrated no electrographic or physiologic seizure activity.

### Example I

This example is to determine whether PDT causes any functional impairment in animal epileptic model. Specifically in this study, we examined the effects of PDT on specific brain regions, using a well established animal model of epilepsy.

To assess behavioral outcome, thirty-one Sprague-Dawley rats were placed into four groups to test the effects of PDT treatment parameters. All animals underwent a behavioral battery that included the Morris Water Maze (MWM), selected to assess one aspect of hippocampal function, spatial learning and memory and incline plane (IP) to screen for motor injury.

The purpose of this study was to determine if PDT causes cortical or hippocampal functional impairment in a rat model of epilepsy and to perform histological studies to analyze the laser effects and localization.

Methods:

Surgical Perforant Path Electrode Implantation:

1. Male Sprague-Dawley rats (275 g) had bipolar stimulating electrodes stereotactically implanted under surgical anesthesia at 7.4 mm posterior to Bregma and 4.1 lateral to the midline through a 1.5 mm craniotomy.

2. Stimulation of electrodes implanted into the perforant path induces seizure activity in the rat model of epilepsy.

Kindling:

1. Kindling is a well-characterized animal model of epileptogenesis, the process by which cells become epileptic (Sutula, 1991).

2. Brief, low-intensity stimulation through bipolar electrodes induces an epileptic progression from focal, to complex, to fully generalized tonic-clonic seizures in rats.

3. Using observable behavioral criteria, seizures were classified into progressive stages (Racine, 1972).

4. With continued stimulation animals will develop spontaneous seizures, making kindling an excellent experimental model of temporal epilepsy.

5. The seizure propagation seen in kindling parallels that in humans with secondarily generalized partial epilepsy.

Drug Administration and Laser Application (PDT):

1. 5-Aminolevulinic acid (ALA) is a photosensitizing agent which is activated by exposure to laser energy producing cytotoxic photoproducts (singelt oxygen) which can lead to apoptosis and necrosis in areas where it has accumulated.

2. Epileptic neurons have been shown by the present invention to take up increased amounts of ALA based on the fact that ALA is effected by pH, metabolic activity and blood brain barrier permeability.

3. ALA (400 mg/Kg) was injected intravenously through a femoral vein catheter.

4. Four hours post drug administration animals were anesthetized and stereotactically positioned.

5. A 4 mm diameter was created and a laser was positioned 2.3 mm from the surface of the brain.

6. Laser light was focused on the brain for 10 minutes at a wavelength of 635 nanometers.

Behavioral Study Design:

4 groups with 8 animals per group

| | |
|---|---|
| Group A: | naive control |
| Group B: | craniectomy control, sham laser application |
| Group C: | implanted, kindled, ALA injection, one induced seizure |
| | (generalized), crani, sham laser |
| Group D: | implanted, kindled, ALA injection, one induced seizure, crani, |
| | laser treatment |

Behavioral Tests:

Morris Water Maze (MWM):

1. This test was selected because it has been used extensively to assess and compare memory and learning in rodents. The target of our therapy, the hippocampus, is believed to be responsible for spatial learning and memory.

2. This task was chosen so that we would be able to determine if excessive functional damage occurred as a result of PDT.

3. The MWM requires no pre-training period, can be accomplished in a short amount of time, and performance can be compared between and within groups.

4. All animals participated in the MWM test which was conducted over 5 consecutive days.

Incline Plane (IP):

1. This test was selected because it can evaluate bilateral grip strength and we used this test to screen for any motor and/or coordination deficiencies resulting for the surgical craniectomy or laser effect on the cortex.

2. Any sign of hemiparesis may indicate undesired cortical injury.

3. This test involves the initial placement of the animal at a 65 degree angle to the horizontal. The flat plane can move up or down at intervals of 5 degrees, until they are at an angle where they can hold their position without sliding down. Then the opposite side is tested. Animals are evaluated on this task on days 1-4 before going on to the MWM.

Results:

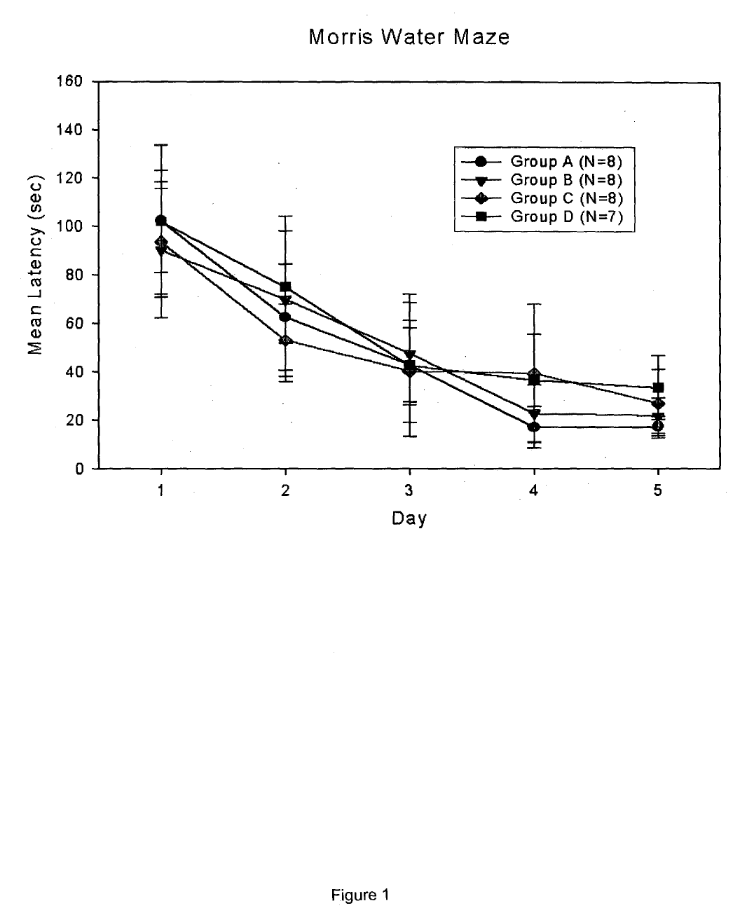

Statistical analysis of performance on the MWM task indicated that there were no significant differences between animals that received laser treatment and control animals [F(3,27)=0.920, p=0.445, between groups] (See also FIG. 1).

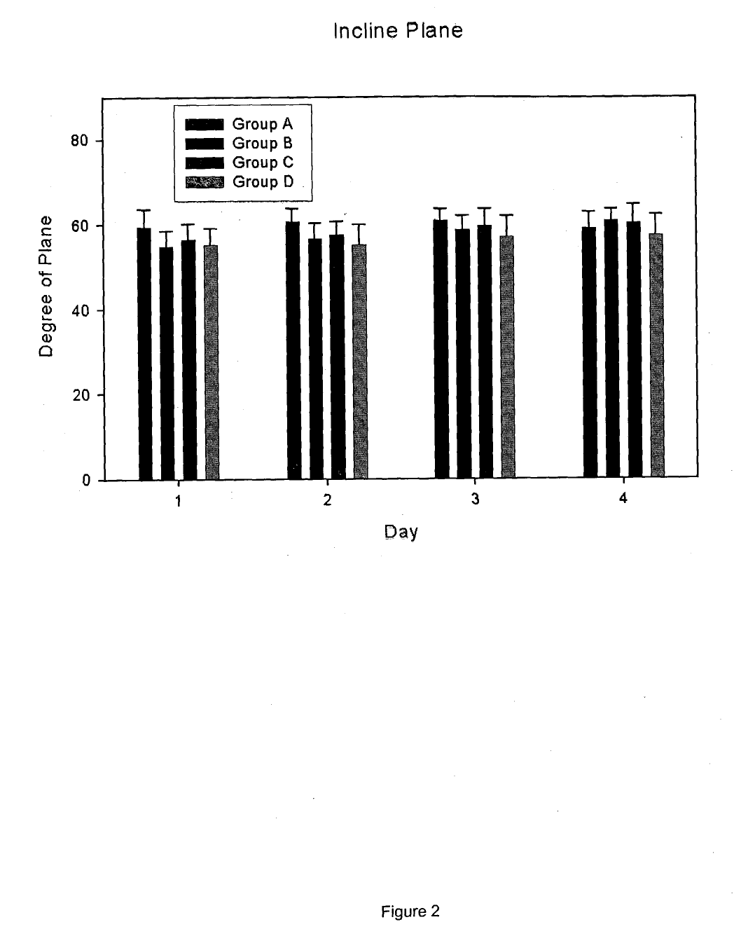

In addition, there was no difference between groups on the incline plane assessment [F(3,27)=1.616, p=0.209, between groups] which demonstrates that the treatment did not influence motor performance; may produce elevations in intracellular [Na+] in vulnerable astrocytes populations sufficient to cause reversal of the Na+/Ca++ exchanger (See also FIG. 2).

Image of hippocampus also demonstrates that animals in Group C showed no pathologies related to craniotomy.

Conclusions:

Photodynamic therapy may have caused hippocampal cellular loss, however no significant hippocampal or cortical functional impairment was identified as measured by the MWM and IP tests.

### Example II

This example is directed to fluorescent labeling of hippocampal cells in epileptic animal models using aminoluvelinic acid (ALA).

ALA is a component of the heme synthesis pathway and is a FDA approved drug used in photodynamic therapy for certain tumors (Peng and Warloe, 1997). It is converted in the mitochondria to protoporphyrin IX (PpIX), a fluorescent and photosensitizing agent when excited with certain wavelengths of light.

We demonstrated in this example that ALA was selectively taken up by cells involved in seizure generation, in vivo, and converted to PpIX.

Currently, electroencephalogram (EEG) is used to localize seizure initiation sites in epileptic brains because tissue that is involved in seizure generation often does not appear different from normal tissue. A fluorescent labeling technique would provide clinicians with another and perhaps more specific method of defining the area of seizure generation.

Localization of PpIX fluorescence to cells involved with seizure generation in rats after ALA infusion could be a very effective way of visualizing cells involved in seizure generation in vivo. This can lead to improved treatment of epilepsy in humans.

Methods:

Kindling: 250 gm male Sprague-Dawley rats were stereotactically implanted with electrodes at 7.4 mm posterior from bregma, 4.1 mm lateral to midline, and 3.3 mm ventral to skull. Rats then underwent the perforant path kindling paradigm to establish a hippocampal epileptic focus (Michalakis, M., et al., 1998). Briefly, animals received a threshold stimulus each day until they achieved three consecutive stage 5 seizures. They were considered fully kindled at that point (Racine, R., 1972).

Experimental design: We compared PpIX fluorescence in four groups of Sprague-Dawley rats:

| | Stereotaxic | | | Induced |
|---|---|---|---|---|
| Group | Implantation | Kindled | 5-ALA | Seizures |
| A | No | No | No | N/A |
| B | Yes | No | 400 mg/Kg IV | N/A |
| C | Yes | Yes | 400 mg/Kg IV | 0 |
| D | Yes | Yes | 400 mg/Kg IV | Yes |

Quantification of Fluorescence Brains were flash frozen in isopentane at −40° C. 4 hours after treatment and sliced into 40 μm sections. The slices were excited at 405 nm and the red wavelength emissions were captured with a SPOT digital camera. Images were analyzed using a Texas Red filter and then converted to gray scale using Image-Pro software. Relative fluorescence was quantified as mean optical density of the gray scale images.

### SUMMARY OF RESULTS

Preliminary data with a small number of animals suggest that fully kindled rats with elicited seizures show greater ALA uptake in hippocampal cells compared with both fully kindled rats without elicited seizures and the implanted controls. Our initial results also suggest that kindled rats without elicited seizures show more fluorescence than implanted controls.

### CONCLUSIONS

Our experiments indicate that cells involved in seizure generation take up more ALA compared to non-seizure controls. Kindling itself also enhance ALA uptake since a kindled animal without seizures showed more uptake than non-kindled controls. These initial results provide a first step towards using ALA in visualization of epileptic foci and applying photodynamic therapy to epilepsy.

### EXAMPLE III

This example is directed to using ALA to demonstrate that cells associated with epileptic seizure preferentially uptake ALA and can be eliminated by activating the photoactive compound induced by ALA.

The ubiquitous intracellular compound, 5-Aminolevulinic acid (ALA) can be converted to the photoactive molecule, protoporphyrin IX (PpIX), part of the heme biosynthetic pathway (Lucroy, M., 1999). Excess exogenous ALA usually bypasses the rate-limiting step of the heme pathway and leads to an excess intracellular accumulation of PpIX. (Boogert, J., 1998)

Conjugated PpIX molecules usually can be excited by violet light (400 nm) and emit red light (635 nm) that can be visualized by fluorescent microscopy. In vivo, PpIX can be activated by exposure to laser energy producing cytotoxic photoproducts, mainly singlet oxygen species which lead to tissue apoptosis and necrosis. Selective accumulation of ALA-induced PpIX in various types of tumor tissues has been demonstrated, and the photosensitizing properties of PpIX post ALA administration are utilized in photodynamic therapy (PDT) as a treatment for different cancer types.

In brain, ALA-induced PpIX has been shown to concentrate more readily in the cortex and in tumor than in white matter, suggesting promising results for selective tumor destruction via PDT (Lilge, L. and BC Wilson, 1998). Since ALA can cross the blood-brain barrier as evidenced by increased PpIX concentrations in normal brain tissue after ALA administration (Hebeda, K., 1995), PDT has been used intraoperatively for the treatment of malignant brain tumors, with laser light delivered fiberoptically into the tumor bed.

Our work using the rat kindling model of epilepsy has suggested that neurons in an epileptic brain take up increased amounts of ALA as compared to normal neurons. We have seen qualitatively increased levels of PpIX fluorescence in epileptic regions of the hippocampus. Fluorescence microscopy with a Texas Red filter to visualize emitted light at 635 nm, was used to identify PpIX positive cells.

We have also performed a quantitative study to measure PpIX fluorescence as a function of ALA uptake using spectrofluorometry. Quantitative comparisons of PpIX fluorescence in frontal cortex and hippocampus were made between kindled and non-kindled animals. Additionally, the role of seizure activity in mediating ALA uptake (and PpIX synthesis) was evaluated by comparing two groups of kindled animals—one that was stimulated to evoke seizures, and the other that received no stimulation after they were kindled.

Animals were randomly assigned to one of the three groups: Group A animals were implanted, non-kindled controls; Group B animals were implanted and fully kindled, but no seizures were induced following ALA injection; Group C animals were implanted and fully kindled, and then received additional stimulation (which induced seizures) following ALA injection. Group A represents the implanted control group; Group B shows ALA uptake due to the kindling process; and Group C shows the effect of induced seizures on ALA uptake and conversion to PpIX.

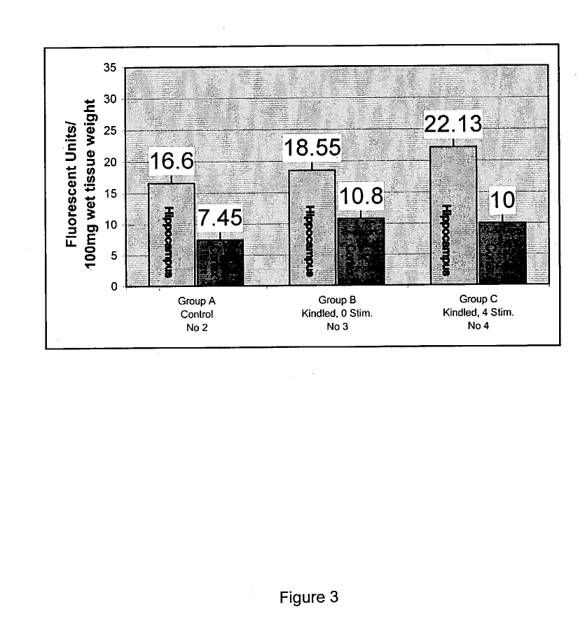

Fluorescence was measured using spectrofluroemetry in tissue homogenates from a total of 16 samples; values were recorded as fluorescent units per 100 mg wet tissue weight (F.U./100 mg) as shown in Table 1. In each group, coronal brain sections through the hippocampus contained more PpIX than sections through the frontal cortex (see also FIG. 3). The animals that were kindled had a higher average PpIX content than the control. Animals that were kindled and stimulated to seizure had the highest average amount of PpIX. Table 1 shows spectrofluoremetric data for rat sample analyzed.

TABLE 1

| | | Frontal Cortex | Ant. Hippocampus |
|---|---|---|---|
| | Rat # | F.U./100 mg | F.U./100 mg |
| Group A | 1302 | 8.2 | 16.6 |
| Implanted | 1303 | 6.7 | X |
| Control | mean | 7.45 | 16.6 |
| n = 2 | | | |
| Group B | 1305 | 8.4 | 21.3 |
| Kindled | 1308 | 11.2 | 15.8 |
| 0 Stimulations | 1365 | 12.8 | X |
| n = 3 | mean | 10.8 | 18.55 |
| Group C | 1300 | 7.1 | 21.5 |
| Kindled | 1306 | 21.5 | 35.9 |
| 4 Stimulations | 1366 | 6.2 | 15.1 |
| n = 4 | 1420 | 5.2 | 16 |
| | mean | 10 | 22.125 |

In our study, Fluorojade histofluorescence analysis demonstrated selective cell death in the CA1 and CA3 regions of the kindled hippocampus 24 hours after laser-induced PDT of ALA-treated animals. The Wilson laboratory at the University of Toronto has previously measured apoptotic cell death at 24 hours following PDT and our Fluorojade results are consistent with their findings. Lilge et al. (2000) have shown that necrotic and apoptotic cell death are a function of the laser exposure (a variable easy to control) and not extent of ALA uptake or conversion.

In our study, animals completing the photodynamic therapy protocol recovered from surgery and underwent behavioral testing using the beam walk test to assess paresis and the Morris Water Maze to assess hippocampus related learning. These animals demonstrated no hemiparesis or learning impairment when compared to historic controls from our laboratory. Following this behavior assessment, animals resumed daily stimulations in the kindling protocol; PDT animals demonstrated no seizure activity or significant after discharges over 1 week of daily stimulation.

### EXAMPLE IV

This example is directed to several studies that are useful for the application of the present invention.

The first study addresses alterations in kindling following photodynamic therapy (See also FIG. 4 for summary of procedures). Four groups with 8 animals each are included in the study design. All animals undergo electrode implantation allowing both stimulation and EEG recording of seizures. In groups A, B and C, the animals undergo perforant path kindling until they are fully kindled to 3 consecutive Stage 5 seizures. Group D has EEG monitoring, but no stimulation is performed. All four groups receive 400 mg/kg ALA IV through surgically implanted femoral catheters to assure consistent drug delivery and uptake and for animal comfort.

Groups A, B and C have additional seizures induced four hours following the ALA injection which is the time point of maximal uptake of ALA into brain tissue (Lilge, L., et al. 2000). Group A undergoes right temporal craniotomy followed by 10 minute laser therapy using a Spectraphysics Laser Model 2500 Argon-pumped tunable dye laser (635 nanometers wavelength at a power density of 200 milliwatts per centimeter squared). Group B also undergoes right temporal craniotomy, but with sham laser therapy for 10 minutes. Group C has sham surgery and sham laser therapy with comparable anesthetic techniques and procedure timing. Group D, which had never before been stimulated through the implanted electrode, undergo right temporal craniotomy and 10 minute laser therapy using the paradigm for Group A. All animals have a 7 day recovery period and then all animals return to the electorphysiologic kindling process.

Groups A, B and C, which had previously been fully kindled, now receive stimulation at the same stimulus intensity which had previously evoked seizures. Stimulation is given each day for 30 days or until 3 consecutive Stage 5 seizures are again achieved. Group D is kindled according to the de novo kindling. The stimulation thresholds for Group D are measured and compared to thresholds for the other groups. Kindling proceeds with daily stimulations until each animal becomes fully kindled or for 30 days. This Group D will show if kindling thresholds are altered by PDT, and if this therapy effects the kindling process. Animals are sacrificed seven days following their third Stage 5 seizure or after the 30 day experimental period for animals who do not fully kindle.

All animals are euthanized and perfused with cold phosphate buffered saline. Whole brain is extracted and flash frozen for histopathology. 20 micron sections cut by cryostat are prepared for Cresyl Violet and Fluorojade staining. Cresyl Violet allows evaluation of necrosis and cell damage following therapy. Fluorojade, a marker of apoptotic cell death, will also be assessed although may not reliably reflect apoptotic cell death when evaluated at this late time point. Cell counting is performed using unbiased stereological techniques.

The second segment of the study is designed to assess long-term effects of photodynamic therapy. A small group of animals (n=6 per group) undergoing the same experimental protocol as described for Group A for the first study, receive daily low level stimulations for one year, or until animals are again fully kindled to three consecutive Stage 5 seizures. This component of the project will follow up on findings from the first study to determine at what point animals can rekindle or if the changes resulting from photodynamic therapy are retained for at least one year.

Animals are sacrificed seven days following the third Stage 5 seizure or at one year and seven days following resumption of kindling (if they are unable to be rekindled following PDT). Tissue is prepared for histology and stereology and analyzed following Cresyl Violet and Flourojade staining.

The third segment of the study is designed to assist in interpreting histological and stereologic results of the first and second study. For this segment, three groups of eight animals have electrodes implanted and undergo perforant path kindling. These groups, E, F, and G are analogous to groups A, B and C in the first study. A fourth group, Group H is implanted but not kindled prior to ALA injection, craniotomy, and laser therapy, matching Group D in the first study.

These animals are sacrificed 24 hours following completion of laser therapy. Following perfusion and brain extraction, tissue is prepared for histology. Because Fluorojade selectively marks apoptotic cells, and since apoptosis associated with photodynamic therapy is likely to be maximal within one day following treatment, this 24 hour sacrifice group is important to determine the mechanism of cell death associated with photodynamic therapy for epilepsy. While Fluorojade may be very informative at this time point, it will obviously not identify cells that are lost during a delayed cell death process. This may best be evaluated in the delayed sacrifice group from the first study.

Methods and Procedures Suitable for the Studies

Subjects

Adult male Sprague-Dawley rats weighing 275 g at the time of surgery are individually housed with food and water available ad libitum. Each animal is handled daily. Experiments are conducted in the light portion of the 12:12 hour light/dark cycle.

Surgical Perforant Path Electrode Implantation

Electrode implantation into the perforant path is one way to induce seizure activity in the rat model of epilepsy. Perforant path kindling allows electrical stimulation to reach the hippocampus without physically damaging the hippocampal areas of interest. Bipolar electrodes are implanted into the entorhinal cortex, a neuronal pathway which synapses onto neurons in the dentate gyrus of the hippocampus. Granule cells in the dentate then project to the CA3 region of the hippocampus.

All animals are housed and cared for according to the UC Davis animal care standards and the methods and procedures follow an approved animal use protocol. Rats are intubated and surgically anesthetized with isoflurane gas mixed with gaseous oxygen (2 parts) and nitrogen (1 part). Bipolar stimulating electrodes are sterotactically implanted through a 1.5 mm diameter craniotomy at 7.4 mm posterior to Bregma, 4.1 mm lateral to midline on the left, and 3.3 mm ventral from cranial surface. The electrodes are constructed from two twisted strands of teflon-coated nichrome wire, attached to a female connector pins (P. Mohapel et al. 1997). A 2 mm diameter silver ball placed into the skull serves as both the ground and reference electrode. The electrodes are secured using three stainless steel screws and dental acrylic.

Thresholding

Kindling begins following a 7-day post-surgical recovery period after the electrode implantation. After discharge thresholds (ADTs) are determined by delivering electrical stimulation consisting of a 1-s train of constant current, symmetrical, biphasic square-wave pulses (1 ms duration, 100 Hz) through the implanted bipolar electrodes. These pulses are delivered at an initial intensity of 10 μA and increased to higher intensities by increments of 10 μA at 30 sec intervals until at least a 5-10 sec epileptiform afterdischarge (AD) is evoked. The after discharge threshold (ADT) is therefore defined as the stimulation intensity which first evokes an AD, a brief focal seizure recorded by the electroencephalogram (EEG). A specific ADT is determined for each animal and is used throughout the kindling process. Repeated stimulations (1 per day, 5 times per week) gradually result in the development of epileptic seizures and increased duration of epileptic spiking on the EEG.

In this perforant path kindling paradigm, successive low-level stimulation produces seizures of increasing severity. Using observable behavioral criteria, seizures are classified into the following progressive stages (Racine, R., 1972): short episodes of epileptic spiking without behavioral elements (Stage 0), episodes of blinking (Stage 1), episodes of chewing/nodding (Stage 2), forelimb clonus (Stage 3), bilateral forelimb clonus and rearing (Stage 4), and generalized bilateral tonic-clonic convulsions with rearing and falling (Stage 5). Animals are considered “fully kindled” when they experienced three consecutive Stage 5 seizures.

Drug Administration

Once fully kindled, animals are surgically equipped with a femoral vein catheter. ALA is prepared at 300 mg/ml with sterile phosphate buffer and pH adjusted to 6.5 with 6N NaOH. For all groups, 400 mg/Kg body weight is injected intravenously through the femoral vein catheter.

Craniotomy for Laser Treatment

To prepare animals for laser treatment used to activate the photosensitive metabolite of 5-ALA, PpIX, rats are first anesthetized with pentobarbital, delivered via a femoral cannula. Anesthetized rats are then placed in the stereotaxic frame, and warmed with a heating pad. A craniotomy is made in the left temporal region, just dorsal to external auditory meatus, ventral to the temporalis muscle insertion, and posterior to the zygomatic arch. The dura is left intact. A black plastic shield with 4 mm aperture is attached to the skull around the craniotomy. The Spectraphysics Laser Model 2500 Argon-pumped tunable dye laser of 635 nanometers wavelength and powered density of 200 milliwatts per centimeter squared is focused on the 4 mm aperture for a total of 10 minutes. Following laser therapy the plastic shield is removed, scalp is sutured, and animals are kept warm and hydrated until they are fully recovered from the anesthesia.

Sacrifice and Tissue Collection

All animals are anesthetized with excess chloralhydrate and perfused in subdued light with 100 ml cold, phosphate buffered saline. The whole brain is extracted in parallel with a muscle specimen. Tissues are immediately flash-frozen in cooled isopentane for 30 seconds, then stored in a-70 degrees Celsius in an ultra cold freezer until being sectioned on the cryostat.

Cryostat Sectioning

Cryostat sectioning and tissue collection occurs in subdued light to reduce photobleaching. Overhead laboratory lights are turned off and the working area is illuminated using a non-fluorescent light source. The cryostat is maintained between −15 and −20 degrees Celsius throughout the tissue preparation and sectioning. Beginning at the anterior hippocampal regions corresponding to 1.6 mm posterior to bregma and ending at the posterior aspect of the hippocampus (6.3 mm posterior to bregma) brains are sectioned at 20 μm. Every other section is mounted, 3 per subbed slide, and to be analyzed with Cresyl Violet and Fluorojade staining.

Cresyl Violet Staining

Cryostat-prepared brain sections are slide-mounted, air-dried (overnight), rinsed in distilled water (10 s), then immersed in Cresyl Violet solution (12 ml of 1% stock solution in 100 ml water) for 30 min. Slides are rinsed, dehydrated through alcohols and xylene, and coverslipped.

Fluorojade Histochemistry

Fluorojade (Histo-Chem, Inc) staining will be carried out on 20 micron cryostat-prepared brain sections. Tissue sections are mounted onto gelatinized slides and allowed to dry at room temp. Using a staining rack, slides are immersed in 100% EtOH (3 min), in 70% EtOH (1 min), in distilled water (1 min), and then in 0.06% solution of potassium permanganate (15 min, shaking gently). Slides are rinsed in distilled water, and then immersed in a 0.001% Fluoro-Jade solution (away from light, 30 min, shaking gently) following steps carried out in dim light). Still in dim light, slides are then rinsed, dried, dehydrated (xylene), and coverslipped. Stained slides, viewed with a fluorescence microscope (FITC filter), reveal apoptotic cells as brightly green.

Stereology

Unbiased stereological techniques are used to estimate cell damage/loss associated with the PDT. The focus can be particularly on the hippocampus. Hippocampal volume can be calculated by the Cavalieri method. This method estimates the volume of a structure by measuring the area of the structure in a number of evenly spaced “two-dimensional” sections.

To carry out this measure, evenly spaced sections that encompass the entire hippocampus is sampled using a systematically random collection. Using a random start point from the first appearance of anterior hippocampus, measurements from every 10^{th} section can be obtained, thus ensuring that each level of hippocampus has an equal probability of being analyzed.

Hippocampal area is estimated with suitable precision by applying to each section a point grid with a known area associated with each point (a/p). Hippocampal volume (V) is then be calculated using the formula: V=(T)·(a/p)·ΣPᵢ; where T distance between sections, P=points landing on the hippocampus on the ith section. The grid generation and volume calculations are performed with Stereologer software on an IBM PC system connected to a Nikon E600 microscope with motorized xyz stage controller (ASI MS-2000).

Unbiased cell counting is performed using the optical fractionator stereological method. This method is based on the principle that the number of cells in a whole object can be accurately estimated by counting the number of cells in a known fraction of the object. The volume of the area of interest is first calculated by the Cavalieri principle described above. The NeuroZoom software divides the area of interest on each slide into “dissectors” which are small volumes of tissue from which the cell counts are made. It is only necessary to count approximately 10% of the dissectors to arrive at accurate estimates of the number of cells in the entire object. The software randomly selects the dissectors to be counted.

### BIBLIOGRAPHIC REFERENCES

Bittar, R. G., et al., Epilepsia, 40(2), 170-178.

Boogert, J et al., Journal of Photochemistry and Photobiology. B, Biology, Jun. 15, 1998, 44 (1):29-38.

Hebeda, K. et al., Thesis: Photodynamic theapy of brain tumors, Amsterdam, 1995, pp. 111-131.

Kostron, H; et al., J Photochemistry Photobiology, 1996, B36: 157-168.

Lilge, L; et al., British Journal of Cancer, 2000 83(8)m 1110-1117.

Lilge, L; et al., Journal of Clinical Laser Medicine and Surgery, 1998 April, 16(2):81-91.

Lucroy, M D; et al., Journal of Veterinary Research, 1999 November, 60(11):1364-70.

Michalakis, M., et al. Brain Research 793 (May 18, 1998): 197-211.

Mohapel, P; et al., Brain Research, Dec. 5, 1997, 778(1):186-93.

Peng, Q; et al., Cancer, Jun. 15, 1997, 79(12):2282-308.

Racine, R J. et al., Electroencephalography and Clinical Neurophysiology 32 (1972): 269-74.

Racine, R. Electroencephalography and Clinical Neurophysiology 32 (1972): 281-294.

Raskin, N. Harrison's Principles of Internal Medicine. (14^{th} edition) New York: McGraw-Hill Health Professions Division, (1998), 2331-2324 (vol.II).

Wyler, A and Vossler, D. Current Diagnosis 9 (9^{th} edition) Philadelphia: W. B. Saunders a division of Harcourt Brace and Company, (1997), 857-859.

Although the invention has been described with reference to the presently preferred embodiment, it should be understood that various modifications can be made without departing from the spirit of the invention. Accordingly, the invention is limited only by the following claims.

What is claimed is:

1. A method of triggering a cell death process in a cell associated with a seizure condition comprising exposing the cell to a photoactive compound and irradiating the cell with an energy source comprising at least one wavelength capable of being absorbed by the photoactive compound, whereby triggering a cell death process in the cell.

2. The method of claim 1, wherein the cell is in a human.

3. The method of claim 2, wherein the cell is irradiated after undergoing at least one episode of the seizure condition.

4. The method of claim 1, wherein the seizure condition is epileptic seizure.

5. The method of claim 1, wherein the seizure condition is partial seizure or primary generalized seizure.

6. The method of claim 1, wherein the photoactive compound is selected from the group consisting of 5-aminolevulinic acid, protoporphyrin IX, Photofrin, chloroaluminium phthalocyanine, Tin Ethyl Etiopurpurin, and meta-tetra (hydroxyphenyl)chlorin.

7. The method of claim 1, wherein the cell is irradiated through non-surgical means.

8. The method of claim 1, wherein the cell is irradiated through a surgical procedure.

9. The method of claim 1, wherein the energy source is a laser.

10. The method of claim 1, wherein the cell death process is apoptosis.

11. A method of treating a seizure condition comprising administering to a subject in need of such treatment an effective amount of a photoactive compound and irradiating cells associated with the seizure condition with an energy source comprising at least one wavelength capable of being absorbed by the photoactive compound.

12. The method of claim 11, wherein the subject is human.

13. The method of claim 11, wherein the cells associated with the seizure condition are irradiated after the subject undergoes at least one episode of seizure.

14. The method of claim 11, wherein the seizure condition is epileptic seizure.

15. The method of claim 11, wherein the seizure condition is partial seizure or primary generalized seizure.

16. The method of claim 11, wherein the photoactive compound is selected from the group consisting of 5-aminolevulinic acid, protoporphyrin IX, Photofrin, chloroaluminium phthalocyanine, Tin Ethyl Etiopurpurin, and meta-tetra (hydroxyphenyl)chlorin.

17. The method of claim 11, wherein the cells associated with the seizure condition are irradiated through non-surgical means.

18. The method of claim 11, wherein the cells associated with the seizure condition are irradiated through a surgical procedure.

19. The method of claim 11, wherein the energy source is a laser.

20. The method of claim 11, wherein irradiating cells triggers a cell death process in the cells associated with the seizure condition.

21. The method of claim 11, wherein irradiating cells triggers apoptosis in the cells associated with the seizure condition.

22. A method of labeling a cell associated with a seizure condition comprising exposing a population of cells to a photoactive compound, whereby a cell associated with a seizure condition is labeled by up-taking the photoactive compound.

23. The method of claim 22, wherein the population of cells are in a brain tissue.

24. The method of claim 22, wherein the population of cells are hippocampal cells.

25. The method of claim 22, wherein the population of cells are in a brain of a subject undergoing a treatment for the seizure condition.

26. The method of claim 25, wherein the population of cells are exposed to the photoactive compound before the subject undergoing the treatment for the seizure condition.

27. The method of claim 25, wherein the treatment for the seizure condition is to surgically remove cells associated with the seizure condition.

28. The method of claim 25, wherein the treatment for the seizure condition is to trigger a cell death process in the cell that is labeled by up-taking the photoactive compound.

29. The method of claim 22, wherein the photoactive compound is selected from the group consisting of 5-aminolevulinic acid, protoporphyrin IX, Photofrin, chloroaluminium phthalocyanine, Tin Ethyl Etiopurpurin, and meta-tetra (hydroxyphenyl)chlorin.

30. The method of claim 22, wherein the photoactive compound is conjugated to a detectable entity.

31. The method of claim 22, wherein the photoactive compound is conjugated to an imaging entity.

32. The method of claim 22, wherein the photoactive compound is conjugated to a fluorescent entity.

33. The method of claim 22, wherein the photoactive compound induces a fluorescent entity.

34. The method of claim 22, wherein the seizure condition is epileptic seizure.

35. The method of claim 22, wherein the seizure condition is partial seizure or primary generalized seizure.

36. A kit comprising a photoactive compound and an instruction for using the photoactive compound to trigger a cell death process in cells associated with a seizure condition.

37. The kit of claim 36, wherein the photoactive compound is selected from the group consisting of 5-aminolevulinic acid, protoporphyrin IX, Photofrin, chloroaluminium phthalocyanine, Tin Ethyl Etiopurpurin, and meta-tetra (hydroxyphenyl)chlorin.

38. The kit of claim 36, wherein the cell death process is apoptosis.

39. The kit of claim 36, wherein the seizure condition is epileptic seizure.

40. The kit of claim 36, wherein the seizure condition is partial seizure or primary generalized seizure.

41. A kit comprising a photoactive compound and an instruction for labeling cells associated with a seizure condition.

42. The kit of claim 41, wherein the photoactive compound is selected from the group consisting of 5-aminolevulinic acid, protoporphyrin IX, Photofrin, chloroaluminium phthalocyanine, Tin Ethyl Etiopurpurin, and meta-tetra (hydroxyphenyl)chlorin.

43. The kit of claim 41, wherein the photoactive compound is conjugated with a detectable entity.

44. The kit of claim 41, wherein the photoactive compound is conjugated with an imaging entity.

45. The kit of claim 41, wherein the photoactive compound is conjugated with a fluorescent entity.

46. The kit of claim 41, wherein the photoactive compound induces a fluorescent entity.

47. The kit of claim 41, wherein the instruction is for labeling cells associated with a seizure condition in a subject undergoing a treatment for the seizure condition.

48. The kit of claim 47, wherein the treatment for the seizure condition is to surgically remove cells associated with the seizure condition.

49. The kit of claim 41, wherein the seizure condition is epileptic seizure.

50. The kit of claim 41, wherein the seizure condition is partial seizure or primary generalized seizure.

51. A neural model system useful for studying a seizure condition wherein cells associated with the seizure condition are selectively reduced by being exposed to a photoactive compound and irradiated by an energy source comprising at least one wavelength capable of being absorbed by the photoactive compound.

52. The neural model system of claim 51, wherein the neural model system is selected from the group consisting of a neural cellular tissue culture, brain slice model, and an animal model.
